(19) United States
(12) Patent Application Publication
(10) Pub. No.: US 2015/0297481 A1
(43) Pub. Date: Oct. 22, 2015

(54) COSMETIC COMPOSITION AND PROCESS FOR TREATING HUMAN KERATIN FIBERS

(71) Applicants: Arno Wahler, Maisons-Laffitte (FR); Daisuke Misu, Yokohama-shi (JP); Maxime De Boni, Munbai (IN)

(72) Inventors: Arno Wahler, Maisons-Laffitte (FR); Daisuke Misu, Yokohama-shi (JP); Maxime De Boni, Munbai (IN)

(73) Assignee: L'OREAL, Paris (FR)

(21) Appl. No.: 14/377,981

(22) PCT Filed: Feb. 14, 2012
(86) PCT No.: PCT/JP2012/053903

§ 371 Date: Jun. 12, 2015

(51) Int. Cl. A61K 8/31; A61Q 5/08; A61Q 5/10; A61K 8/25; A61K 8/891; A61K 8/41; A61K 8/22; A61K 8/04; A61K 8/29

(52) U.S. Cl. CPC A61K 8/31; A61K 8/04; A61Q 5/08; A61Q 5/10; A61K 8/29; A61K 8/891; A61K 8/41; A61K 8/22; A61K 8/25; A61K 8/415; A61K 8/411; A61K 2800/30; A61K 2800/43; A61K 2800/592

(57) ABSTRACT

The present invention relates to a cosmetic composition in the form of an emulsion, comprising: (a) a continuous phase; (b) at least one dispersed phase, in the continuous phase, different from the component (c); (c) water-insoluble particles; (d) at least one water-soluble salt and (e) at least one alkaline agent, wherein (d) and (e) are distinct compounds and wherein the composition optionally comprises at least one surfactant in an amount of 1 wt % or less relative to the total weight of the composition. The composition is intended for bleaching or colouring human keratin fibres.

### TECHNICAL FIELD

The present invention relates to a cosmetic composition in the form of a dispersion, in particular for bleaching or coloring human keratin fibers such as hair, and a process for treating human keratin fibers, in particular for bleaching or coloring human keratin fibers.

### BACKGROUND ART

For cosmetic compositions for bleaching or dyeing human keratin fibers such as hair, an alkaline agent is a key material to achieve bleaching of the hair together with hydrogen peroxide. For a long time, ammonia has been used as the alkaline agent.

In addition to higher performance of products for bleaching or coloring hair, consumers of such products are more and more sensitive to the usage quality of the products. From the view point of usage quality, for example, malodor from ammonia, which is typically contained as an alkaline agent in conventional hair bleaching or dyeing products, and the like, can be regarded as a significant drawback.

In order to reduce the problems occurring with the pungent odor of ammonia, it has been proposed to replace totally or partially this alkaline agent in a cosmetic composition by another one such as monoethanolamine. However, the consequence of this modification is a decrease of the bleaching or coloring efficiency of the composition.

Moreover, the usual dyeing or bleaching compositions are in the form of emulsions, comprising surfactants/emulsifiers to guaranty their physical stability.

However, emulsifiers may have potential problems such as stimulating the skin and poor feeling to the touch (e.g., dry feeling). In some instances, they may cause a decrease of the efficiency of the composition, such as for instance a decrease of the uptake of the dyes.

### DISCLOSURE OF INVENTION

An objective of the present invention is to provide a cosmetic composition in the form of a dispersion, in particular for bleaching or dyeing human keratin fibers such as hair, which can have higher cosmetic effects such as bleaching ability even if the cosmetic composition comprises a small amount of surfactant(s).

Another objective of the present invention is to provide a cosmetic composition in the form of a dispersion, in particular for bleaching or dyeing human keratin fibers such as hair, which can comprise fatty material(s) without any conventional surfactant or with only a small amount of a conventional surfactant, in order to avoid or reduce any disadvantages such as stimulating the skin and poor feeling to the touch caused by the presence of the conventional surfactant.

The above objectives of the present invention can be achieved by a cosmetic composition in the form of a dispersion, comprising:

(a) a continuous phase;
(b) at least one dispersed phase, in the continuous phase, different from the component (c);
(c) water-insoluble particles;
(d) at least one water-soluble salt; and
(e) at least one alkaline agent,
wherein
(d) and (e) are distinct compounds; and
the composition optionally comprises at least one surfactant in an amount of 1 wt % or less relative to the total weight of the composition.

It is preferable that the continuous phase comprises water, and the dispersed phase comprises at least one fatty material.

It is preferable that the fatty material is in the form of a liquid at ambient temperature and under atmospheric pressure.

It is preferable that the fatty material is selected from the group consisting of oils of animal or plant origin, synthetic glycerides, fatty esters other than animal or plant oils and synthetic glycerides, fatty alcohols, fatty acids, silicone oils, and aliphatic hydrocarbons.

It is more preferable that the fatty material is chosen from aliphatic hydrocarbons and liquid fatty alcohols, preferably from aliphatic hydrocarbons. In particular, the fatty material may be mineral oil.

The amount of the fatty material(s) in the cosmetic composition may be 50% by weight or less, particularly 40% by weight or less, more preferably 30% by weight or less, and even more preferably 20% by weight or less, relative to the total weight of the composition.

It is preferable that the water-soluble salt is selected from water-soluble inorganic salts, preferably from silicates, metasilicates, carbonates, hydrogenocarbonates, phosphates, sulfates of alkaline metals, halides of ammonium, halides of alkaline metals, and their mixtures.

It is more preferable that water-soluble salt is selected from water-soluble alkaline salts, further more preferably from silicates, metasilicates of alkaline metals, and their mixtures.

The amount of the water-soluble salt(s) in the cosmetic composition may be from 0.01 to 50% by weight, preferably from 0.1 to 40% by weight, and more preferably from 0.5 to 30% by weight, relative to the total weight of the composition.

It is preferable that the water-insoluble particle comprises at least one inorganic material.

It is preferable that the inorganic material is selected from metal oxides, more preferably aluminum or transition metal oxides, and silicon oxides, these compounds being optionally surface-treated, and mixtures thereof.

The water-insoluble particle may have at least one hydrophobic coating.

The amount of the water-insoluble particle(s) in the cosmetic composition may be from 0.01 to 30% by weight, preferably from 0.1 to 20% by weight, and more preferably from 0.5 to 10% by weight, relative to the total weight of the composition.

The alkaline agent may be chosen from inorganic compounds such as ammonia, alkaline metal hydroxides; alkaline earth metal hydroxides; alkaline metal (hydrogeno)carbonates; alkaline earth metal(hydrogeno)carbonates; and alkaline metal (meta)silicates, and mixture thereof.

The alkaline agent may not be a water-soluble salt.

The alkaline agent may be chosen organic alkaline agents such as monoamines and derivatives thereof; diamines and derivatives thereof; polyamines and derivatives thereof; amino acids and derivatives thereof; oligomers of amino acids and derivatives thereof; polymers of amino acids and derivatives thereof; urea and derivatives thereof; and guanidine and derivatives thereof; and their mixtures, and preferably non-volatile organic alkaline agents.

The alkaline agent may be a non-volatile agent.

The alkaline agent may be selected from alkanolamines, preferably monoethanolamine.

The amount of the alkaline agent(s) may be from 0.01 to 15% by weight, preferably 0.1 to 10% by weight, and more preferably 1 to 5% by weight, relative to the total weight of the composition.

The cosmetic composition according to the present invention may further comprise at least one thickening agent.

The present invention also relates to a process for treating human keratin fibers, particularly for dyeing or bleaching these fibers, in which the composition according to the present invention is applied onto the human keratin fibers, generally after mixing with an oxidizing agent, preferably hydrogen peroxide.

The present invention also relates to a process for preparing the cosmetic composition according to the present invention, in which the dispersed phase, the continuous phase, the water-insoluble particles, the water-soluble salt(s) and the alkaline agent are mixed.

### BEST MODE FOR CARRYING OUT THE INVENTION

Pickering reported (J. Chem. Soc. 1907, 9, 2001) a composition stabilized with powder particles. Since then, some dispersions stabilized by powder particles and containing no or only a small amount of emulsifier(s), which are so-called Pickering emulsions, have been proposed.

The inventors performed diligent research and found that a new cosmetic composition based on a Pickering emulsion can be suitably used for cosmetic products, preferably for bleaching or coloring human keratin fibers such as hair.

The cosmetic composition according to the present invention can have superior effects even if the cosmetic composition comprises a smaller amount of surfactant(s). For example, by using the cosmetic composition according to the present invention together with a developer, high bleaching efficiency can be obtained even when the cosmetic composition comprises fatty material(s) in an amount of, for example, less than 40% by weight relative to the total weight of the composition.

Furthermore, since the cosmetic composition according to the present invention comprises no surfactant or may comprise only a small amount of a surfactant, it can avoid or reduce any disadvantages such as stimulating the skin and poor feeling to the touch caused by the surfactant.

Hereinafter, the cosmetic composition according to the present invention will be explained in more detail.

### (1) Form of Dispersion

The cosmetic composition according to the present invention comprises a continuous phase and dispersed phases. Therefore, the continuous phase may be an oily phase when dispersed phase(s) is/are aqueous phase(s), which is the equivalent with a W/O emulsion. On the other hand, the continuous phase may be an aqueous phase when the dispersed phase(s) is/are oily phase(s), which is the equivalent with an O/W emulsion. It is preferable that the cosmetic composition according to the present invention is in the form of the equivalent of an O/W emulsion. In this preferable embodiment, typically, the continuous phase comprises water and the dispersed phases comprise at least one fatty material.

### (2) Fatty Material

The term “fatty material” means an organic compound that is insoluble in water at ordinary temperature (25° C.) and at atmospheric pressure (760 mmHg) (solubility of less than 5 wt %, preferably less than 1 wt % and even more preferably less than 0.1 wt %). The fatty material may contain, in its structure, a sequence of at least two siloxane groups or at least one hydrocarbon-based chain containing at least 6 carbon atoms. In addition, the fatty materials may be soluble in organic solvents under the same temperature and pressure conditions, for instance chloroform, ethanol, benzene, or decamethylcyclopentasiloxane.

In the scope of the present invention, it should be noted that the fatty material(s) does not comprise any C₂-C₃ oxyalkylene units or any glycerolated units.

According to the present invention, two or more fatty materials may be used in combination. Thus, a single type of fatty material or a combination of different types of fatty materials may be used.

The fatty material may be in the form of a liquid or a solid. Here, “liquid” and “solid” mean that the fatty material is in the form of a liquid or a paste (non-solid) or a solid, respectively, at ambient temperature (25° C.) under atmospheric pressure (760 mmHg or 10⁵ Pa). It is preferable that the fatty material be in the form of a liquid or a paste, more preferably in the form of a liquid, at ambient temperature and under atmospheric pressure.

The fatty material may be selected from the group consisting of oils of animal or plant origin, mineral oils, synthetic glycerides, fatty esters other than animal or plant oils and synthetic glycerides, fatty alcohols, fatty acids, silicone oils, and aliphatic hydrocarbons. These fatty materials may be volatile or non-volatile. Preferably, the fatty material(s) is selected from aliphatic hydrocarbons, plant oils, fatty alcohols, fatty esters other than animal or plant oils and synthetic glycerides, or mixtures thereof, and more preferably from aliphatic hydrocarbons.

As examples of aliphatic hydrocarbons, mention may be made of, for example, linear or branched hydrocarbons such as mineral oil (e.g., liquid paraffin), paraffin, vaseline or petrolatum, naphthalenes, and the like; hydrogenated polyisobutene, isoeicosan, polydecenes, hydrogenated polyisobutenes such as Parleam, and decene/butene copolymer; and mixtures thereof.

As examples of other aliphatic hydrocarbons, mention may also be made of linear or branched, or possibly cyclic C₆-C₁₆ lower alkanes. Examples that may be mentioned include hexane, undecane, dodecane, tridecane, and isoparaffins such as isohexadecane and isodecane.

As example of synthetic glycerides, mention may be made of, for instance, caprylic/capric acid triglycerides, for instance those sold by the company Stéarinerie Dubois or those sold under the names Miglyol® 810, 812 and 818 by the company Dynamit Nobel.

As examples of silicone oils, mention may be made of, for example, linear organopolysiloxanes such as dimethylpolysiloxanes, methylphenylpolysiloxanes, methylhydrogenpolysiloxanes, and the like; cyclic organopolysiloxanes such as octamethylcyclotetrasiloxane, decamethylcyclopentasiloxane, dodecamethylcyclohexasiloxane, and the like; and mixtures thereof.

As examples of plant oils, mention may be made of, for example, linseed oil, camellia oil, macadamia nut oil, sunflower oil, apricot oil, soybean oil, arara oil, hazelnut oil, corn oil, mink oil, olive oil, avocado oil, sasanqua oil, castor oil, safflower oil, jojoba oil, sunflower oil, almond oil, grapeseed oil, sesame oil, soybean oil, peanut oil, and mixtures thereof.

As examples of animal oils, mention may be made of, for example, squalene, perhydrosqualene, and squalane.

As examples of the esters of a fatty acid and/or of a fatty alcohol, which are advantageously different from the animal or plant oils as well as the synthetic glycerides mentioned above, mention may be made especially of esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl, or stearyl myristate, hexyl stearate, butyl stearate, isobutyl stearate; dioctyl malate, hexyl laurate, and 2-hexyldecyl laurate.

Still within the context of this variant, esters of C₄-C₂₂ dicarboxylic or tricarboxylic acids and of C₁-C₂₂ alcohols and esters of mono-, di- or tricarboxylic acids, and of C₂-C₂₆ di-, tri-, tetra- or pentahydroxy alcohols may also be used.

The following may especially be mentioned: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, it is preferable to use ethyl, isopropyl, myristyl, cetyl, or stearyl palmitate, 2-ethylhexyl palmitate, 2-octyldecyl palmitate, alkyl myristates such as isopropyl, butyl, cetyl or 2-octyldodecyl myristate, hexyl stearate, butyl stearate, or isobutyl stearate; dioctyl malate, hexyl laurate, 2-hexyldecyl laurate, isononyl isononanoate, or cetyl octanoate.

The composition may also comprise, as fatty esters, sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which contain at least 4 carbon atoms. These sugars may be monosaccharides, oligosaccharides, or polysaccharides.

Examples of suitable sugars that may be mentioned include sucrose (or saccharose), glucose, galactose, ribose, fructose, maltose, mannose, arabinose, xylose, and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or non-conjugated carbon-carbon double bonds.

The esters according to this variant may also be chosen from mono-, di-, tri-, tetraesters, and polyesters, and mixtures thereof.

These esters may be chosen, for example, from oleates, laurates, palmitates, myristates, behenates, cocoates, stearates, linoleates, linolenates, caprates, and arachidonates, or mixtures thereof such as, especially, oleo-palmitate, oleo-stearate and palmito-stearate mixed esters.

It is more particularly preferable to use monoesters and diesters and especially sucrose, glucose, or methylglucose mono- or dioleates, stearates, behenates, oleopalmitates, linoleates, linolenates, and oleostearates.

An example that may be mentioned is the product sold under the name Glucate® DO by the company Amerchol, which is a methylglucose dioleate.

Examples of esters or mixtures of esters of sugar and of fatty acid that may also be mentioned include:
the products sold under the names Crodesta F160, F140, F110, F90, F70, and SL40 by the company Croda, Inc., respectively denoting sucrose palmitostearates formed from 73% monoester and 27% diester and triester, from 61% monoester and 39% diester, trimester, and tetraester, from 52% monoester and 48% diester, triester, and tetraester, from 45% monoester and 55% diester, triester and tetraester, from 39% monoester and 61% diester, triester, and tetraester, and sucrose monolaurate;
the products sold under the name Ryoto Sugar Esters, for example, reference B370 and corresponding to sucrose behenate formed from 20% monoester and 80% di-, tri-, polyester;
the sucrose mono-dipalmito-stearate sold by the company Goldschmidt under the name Tegosoft® PSE.

The fatty material may be at least one fatty acid, and two or more fatty acids may be used. The fatty acids should be in acidic form (i.e., unsalified, to avoid soaps) and may be saturated or unsaturated and contain from 6 to 30 carbon atoms and in particular from 9 to 30 carbon atoms, which is optionally substituted, in particular with one or more hydroxyl groups (in particular 1 to 4). If they are unsaturated, these compounds may comprise one to three conjugated or non-conjugated carbon-carbon double bonds. They are more particularly chosen from myristic acid, palmitic acid, stearic acid, behenic acid, oleic acid, linoleic acid, linolenic acid, and isostearic acid. Preferably, the fatty material is not a fatty acid.

The fatty material may be at least one fatty alcohol, and two or more fatty alcohols may be used.

The term “fatty alcohol” here means any saturated or unsaturated, linear or branched C₈-C₃₀ fatty alcohol, which is optionally substituted, in particular with one or more hydroxyl groups (in particular 1 to 4). If they are unsaturated, these compounds may comprise one to three conjugated or non-conjugated carbon-carbon double bonds.

Among the C₈-C₃₀ fatty alcohols, C₁₂-C₂₂ fatty alcohols, for example, may be used. Mention may be made among these of lauryl alcohol, cetyl alcohol, stearyl alcohol, isostearyl alcohol, oleyl alcohol, behenyl alcohol, linoleyl alcohol, undecylenyl alcohol, palmitoleyl alcohol, linolenyl alcohol, myristyl alcohol, arachidonyl alcohol, and erucyl alcohol, and mixtures thereof. In one embodiment, cetyl alcohol, stearyl alcohol, or a mixture thereof (e.g., cetearyl alcohol), as well as myristyl alcohol, can be used as a solid fatty material. In another embodiment, isostearyl alcohol can be used as a liquid fatty material.

The fatty material may be a wax. Here, “wax” means that the fatty material is substantially in the form of a solid at room temperature (25° C.) under atmospheric pressure (760 mmHg), and has a melting point generally of 35° C. or more. As the waxy fatty material, waxes generally used in cosmetics can be used alone or in combinations thereof.

For example, the wax may be chosen from carnauba wax, microcrystalline waxes, ozokerites, hydrogenated jojoba oil, polyethylene waxes such as the wax sold under the name “Performalene 400 Polyethylene” by the company New Phase Technologies, silicone waxes, for instance poly(C₂₄-C₂₈)alkylmethyldimethylsiloxane, such as the product sold under the name “Abil Wax 9810” by the company Goldschmidt, palm butter, C₂₀-C₄₀ alkyl stearate sold under the name “Kester Wax K82H” by the company Koster Keunen, stearyl benzoate, shellac wax, and mixtures thereof. For example, a wax chosen from carnauba wax, candelilla wax, ozokerites, hydrogenated jojoba oil, and polyethylene waxes may be used. In at least one embodiment, the wax is preferably chosen from candelilla wax and ozokerite, and mixtures thereof.

As fatty, materials, liquid aliphatic hydrocarbons and liquid fatty alcohols are preferable. In particular, fatty materials may be selected from aliphatic hydrocarbons, and mineral oils are preferable.

The amount of the fatty material(s) is not limited but may be 50% by weight or less, particularly 40% by weight or less, more preferably 30% by weight or less, and even more preferably 20% by weight or less, relative to the total weight of the composition.

It is preferable that the amount of the fatty material(s) is less than 40% by weight or less, more preferably 25% by weight or less, relative to the total weight of the composition.

### (3) Water-Insoluble Particles

The cosmetic composition according to the present invention comprises water-insoluble particles which are different from the component (c) such as a fatty material as previously described. For the purposes of the present invention, the term “water-insoluble particle” means a particle with a solubility in water at 25° C. of less than 1 wt %, preferably less than 0.1 wt % and more preferably less than 0.01 wt %, relative to the total weight of the particle, and most preferably with no solubility.

According to the present invention, two or more water-insoluble particles may be used in combination. Thus, a single type of water-insoluble particles or a combination of different type of water-insoluble particles may be used.

The diameter of the water-insoluble particles is not limited but may have a number-average particle size of 200 nm or more. The average particle size of the particles is preferably 250 nm or more, more preferably 300 nm or more, and even more preferably 350 nm or more, and is preferably 2 μm or less, more preferably 800 nm or less, and even more preferably 500 nm or less. The number-average particle size may be measured by dynamic light scattering with, for example, Nicomp Z380.

The water-insoluble particles are preferably in the form of a solid. More preferably, the water-insoluble particles may be powders. The powders may be pigments and/or fillers.

The pigments that have not been surface treated have a solubility in water of less than 0.01 wt %, for example, less than 0.0001 wt % at 20° C., and an absorption ranging from 350 to 700 nm, and in at least one embodiment, an absorption with a maximum in this absorption range.

The pigments that have not been surface-treated, which are referred to hereinbelow as “pigment”, may be an organic pigment. As used herein, the term “organic pigment” means any pigment that satisfies the definition in Ullmann's encyclopedia in the chapter on organic pigments. The organic pigment may be chosen, for example, from nitroso, nitro, azo, xanthene, quinoline, anthraquinone, phthalocyanin, metal complex, isoindolinone, isoindoline, quinacridone, perinone, perylene, diketopyrrolopyrrole, thioindigo, dioxazine, triphenylmethane, and quinophthalone compounds.

The at least one organic pigment may be chosen, for example, from carmine, carbon black, aniline black, melanin, azo yellow, quinacridone, phthalocyanin blue, sorghum red, the blue pigments codified in the Color Index under the references Cl 42090, 69800, 69825, 73000, 74100, and 74160, the yellow pigments codified in the Color Index under the references Cl 11680, 11710, 15985, 19140, 20040, 21100, 21108, 47000, and 47005, the green pigments codified in the Color Index under the references Cl 61565, 61570, and 74260, the orange pigments codified in the Color Index under the references Cl 11725, 15510, 45370, and 71105, the red pigments codified in the Color Index under the references Cl 12085, 12120, 12370, 12420, 12490, 14700, 15525, 15580, 15620, 15630, 15800, 15850, 15865, 15880, 17200, 26100, 45380, 45410, 58000, 73360, 73915, and 75470, and the pigments obtained by oxidative polymerization of indole or phenolic derivatives as described, for example, in French Patent No. 2 679 771.

These pigments may also be in the form of composite pigments as described, for example, in European Patent No. 1 184 426. These composite pigments may be composed, for instance, of particles comprising an inorganic nucleus at least partially coated with an organic pigment and at least one binder to fix the organic pigments to the nucleus.

Other examples may include pigmentary pastes of organic pigments such as the products sold by the company Hoechst under the names:

### Jaune Cosmenyl IOG: Pigment Yellow 3 (Cl 11710);

### Jaune Cosmenyl G: Pigment Yellow 1 (Cl 11680);

### Orange Cosmenyl GR: Pigment Orange 43 (Cl 71105);

### Rouge Cosmenyl R″: Pigment Red 4 (Cl 12085);

### Carmine Cosmenyl FB: Pigment Red 5 (Cl 12490);

### Violet Cosmenyl RL: Pigment Violet 23 (Cl 51319);

### Bleu Cosmenyl A2R: Pigment Blue 15.1 (Cl 74160);

### Vert Cosmenyl GG: Pigment Green 7 (Cl 74260); and

### Noir Cosmenyl R: Pigment Black 7 (Cl 77266).

The at least one pigment may also be chosen from lakes. As used herein, the term “lake” means insolublized dyes adsorbed onto insoluble particles, the complex or the compound thus obtained remaining insoluble during use.

The inorganic substrates onto which the dyes are adsorbed may include, for example, alumina, silica, calcium sodium borosilicate, calcium aluminum borosilicate, and aluminum.

Non-limiting examples of organic dyes include cochineal carmine and the products known under the following names: D&C Red 21 (Cl 45 380), D&C Orange 5 (Cl 45 370), D&C Red 27 (Cl 45 410), D&C Orange 10 (Cl 45 425), D&C Red 3 (Cl 45 430), D&C Red 4 (Cl 15 510), D&C Red 33 (Cl 17 200), D&C Yellow 5 (Cl 19 140), D&C Yellow 6 (Cl 15 985), D&C Green (Cl 61 570), D&C Yellow 10 (Cl 77 002), D&C Green 3 (042 053), and D&C Blue 1 (Cl 42 090).

An additional non-limiting example of a lake is the product known under the following name: D&C Red 7 (Cl 15 850:1).

The at least one pigment may also be a pigment with special effects. As used herein, the term “pigments with special effects” means pigments that generally create a non-uniform colored appearance (characterized by a certain shade, a certain vivacity, and/or a certain lightness) that changes as a function of the conditions of observation (light, temperature, observation angles, etc.). They thus contrast with white or colored pigments that afford a standard uniform opaque, semi-transparent, or transparent shade.

Two types of pigment with special effects exist: those with a low refractive index, such as fluorescent, photochromic, and thermochromic pigments, and those with a high refractive index, such as nacres and flakes.

The at least one pigment may also be chosen from pigments with an interference effect that are not fixed onto a substrate, for instance, liquid crystals (Helicones HC from Wacker), and holographic interference flakes (Geometric Pigments or Spectra fix from Spectratek). Pigments with special effects may also comprise fluorescent pigments, whether these are substances that are fluorescent in daylight or that produce an ultraviolet fluorescence, phosphorescent pigments, photochromic pigments, thermochromic pigments, and quantum dots, sold, for example, by the company Quantum Dots Corporation.

The pigments with special effects may also comprise fluorescent pigments, whether these are substances that are fluorescent in daylight or that produce an ultraviolet fluorescence, phosphorescent pigments, photochromic pigments, and thermochromic pigments.

The pigment may also be an inorganic pigment, in a preferred embodiment. As used herein, the term “inorganic pigment” means any pigment that satisfies the definition in Ullmann's encyclopedia in the chapter on inorganic pigments. Preferably, the inorganic pigments comprise at least one inorganic material. Non-limiting examples of inorganic pigments that are useful in the present invention include metal oxides, in particular, transition metal oxides, such as zirconium oxides, cerium oxides, iron oxides, chromium oxides, manganese violet, ultramarine blue, chromium hydrate, ferric blue, and titanium dioxide. The following inorganic pigments may also be used: Ta₂O₅, Ti₃O₅, Ti₂O₃, TiO, and ZrO₂ as a mixture with TiO₂, ZrO₂, Nb₂O₅, CeO₂, and ZnS.

The pigment may also be a nacreous pigment such as a white nacreous pigment, for example, mica coated with titanium or with bismuth oxychloride, a colored nacreous pigment such as mica coated with titanium and with iron oxides, mica coated with titanium and, for example, with ferric blue or chromium oxide, mica coated with titanium and with an organic pigment as defined above, and also a nacreous pigment based on bismuth oxychloride. Examples of such pigments may include the Cellini pigments sold by Engelhard (Mica-TiO₂-lake), Prestige sold by Eckart (Mica-TiO₂), and Colorona sold by Merck (Mica-TiO₂—Fe₂O₃).

In addition to nacres on a mica support, multilayer pigments based on synthetic substrates such as alumina, silica, calcium sodium borosilicate, calcium aluminum borosilicates, and aluminum, may be useful in accordance with the present disclosure.

As used herein, the term “filler” means a substantially uncolored compound that is solid at room temperature and atmospheric pressure, and insoluble in the various ingredients of the cosmetic composition according to the present invention, even when these ingredients are brought to a temperature above room temperature.

The filler may be chosen from mineral and organic fillers. The filler may be particles of any form, for example, platelet-shaped, spherical, and oblong, irrespective of their crystallographic form (for example lamellar, cubic, hexagonal, and orthorhombic).

Suitable fillers that may be used in the cosmetic composition according to the present invention may include, but are not limited to, titanium dioxide; talc; natural or synthetic mica; silica (or silicon dioxides); kaolin or other insoluble silicates such as clays; polyamides (Nylon®), poly-β-alanine and polyethylene powders; tetrafluoroethylene polymer (Teflon®) powders; lauroyllysine; starch; boron nitride; acrylic acid polymer powders; silicone resin microbeads, for instance “Tospearls®” from the company Toshiba; bismuth oxychlorides; precipitated calcium carbonate; magnesium carbonate and magnesium hydrogen carbonate; hydroxyapatite; hollow silica microspheres such as “Silica Beads SB 700®” and “Silica Beads SB 700®” from the company Maprecos, “Sunspheres H-33®” and “Sunspheres H-51®” from the company Asahi Glass; acrylic polymer microspheres such as those made from crosslinked acrylate copolymer “Polytrap 6603 Adsorber®” from the company RP Scherrer and those made from polymethyl methacrylate “Micropearl M100®” from the company SEPPIC; polyurethane powders such as the hexamethylene diisocyanate and trimethylol hexyl lactone copolymer powder sold under the name “Plastic Powder D-400®” by the company Toshiki; glass or ceramic microcapsules; microcapsules of methyl acrylate or methacrylate polymers or copolymers, or alternatively, vinylidene chloride and acrylonitrile copolymers, for instance, “Expancel®” from the company Expancel; elastomeric crosslinked organopolysiloxane powders such as those sold under the name “Trefil Powder E-506C” by the company Dow Corning; and mixtures thereof.

Among the silicas that are useful in the composition of the invention, mention may be made of crystalline, microcrystalline and non-crystalline silicas.

By way of example, crystalline silicas that may be mentioned include quartz, tridymite, cristobalite, keatite, coesite and stishovite. The microcrystalline silicas are, for example, diatomite.

Among the non-crystalline forms that may be used are vitreous silica and other types of amorphous silicas such as colloidal silicas, silica gels, precipitated silicas and fumed silicas, for instance aerosils, and pyrogenic silicas.

According to the present invention, the above-described water-insoluble particles may be surface treated. The surface treatment can be performed by any conventional process.

For the purposes of the present invention, the surface treatment is such that a surface-treated pigment conserves its intrinsic pretreatment pigmenting properties and a surface-treated filler conserves its intrinsic pretreatment filling properties. For example, the inorganic substrates such as alumina and silica onto which organic dyes are adsorbed are not surface-treated fillers for the purposes of the present invention.

The water-insoluble particles may have at least one hydrophobic coating.

The hydrophobic coating may be formed by treating the water-insoluble particles with a hydrophobic treating agent. The hydrophobic treating agent can be chosen from silicones, such as methicones, dimethicones or perfluoroalkylsilanes; fatty acids, such as stearic acid; metal soaps, such as aluminum dimyristate or the aluminum salt of hydrogenated tallow glutamate, perfluoroalkyl phosphates, perfluoroalkylsilanes, perfluoroalkylsilazanes, poly(hexafluoropropylene oxides), polyorganosiloxanes comprising perfluoroalkyl or perfluoropolyether groups, and amino acids; N-acylated amino acids or their salts; lecithin, isopropyl triisostearyl titanate, and their mixtures.

As the water-insoluble particles, TiO₂ particles coated with at least one hydrophobic coating are preferable. Among the coated TiO₂ particles, mention may be made of:
those coated with polydimethylsiloxane (CARDRE ULTRAFINE TITANIUM DIOXIDE AS provided by the company CARDRE);
those coated with polymethylhydrogenosiloxane (untreated titanium oxide coated with polymethylhydrogenosiloxane sold under the trade name Cosmetic White SA-C47-051-10 by the company MYOSHI);
those coated with perfluoropolymethyl isopropyl ether (CARDRE MICA FHC 70173 OR 70170 CARDRE UF TIO2 FHC provided by the company CARDRE);
those coated with silica (SPHERITITAN AB provided by the company CATALYSTS & CHEMICALS;
those coated with teflon (CS-13997 TEFLON COATED TITANIUM DIOXIDE provided by the company CLARK COLORS); and
those coated with polyester (EXPERIMENTAL DESOTO BEADS provided by the company DESOTO).

As the water-insoluble particles, TiO₂ particles coated with silicone such as polydimethylsiloxane are more preferable.

According to one embodiment of the present invention, the water-insoluble particles may be surface treated with at least one amphiphilic agent, in particular, the above-described water-insoluble particles may be partially or fully surface treated with at least one amphiphilic agent. It is preferable that the particles are partially treated with the amphiphilic agent(s). The water-insoluble particles may be located between the continuous phase and the dispersed phase of the cosmetic composition according to the present invention, to form a Pickering emulsion. The dispersed phases preferably connect with each other via the particles.

The amphiphilic agent can provide particles with both hydrophilic and hydrophobic properties. Preferably, the particles have an amphiphilic surface.

The amphiphilic agent may comprise at least one compound chosen from, for example, amino acids; waxes, for example, carnauba wax and beeswax; fatty acids, fatty alcohols and derivatives thereof, such as stearic acid, hydroxystearic acid, stearyl alcohol, hydroxystearyl alcohol, lauric acid, and derivatives thereof; anionic surfactants; lecithins; sodium, potassium, magnesium, iron, titanium, zinc, or aluminum salts of fatty acids, for example, aluminum stearate or laurate; metal alkoxides; polysaccharides, for example, chitosan, cellulose, and derivatives thereof; polyethylenes; (meth)acrylic polymers, for example, polymethyl methacrylates; polymers and copolymers containing acrylate units; proteins; and alkanolamines.

The particles may be surface treated with a mixture of amphiphilic agents, and/or may be subjected to several surface treatments with amphiphilic agents.

The surface-treated particles may be prepared according to surface-treatment techniques that are well known to those skilled in the art, or may be commercially available in the required form.

Preferably, the surface-treated particles are coated with an organic layer. The organic layer may be deposited on the particles by evaporation of a solvent, chemical reaction between the molecules in the amphiphilic agents, or creation of a covalent bond between the molecules in the amphiphilic agents and the particles.

The surface treatment may thus be performed, for example, by chemical reaction of the amphiphilic agent with the surface of the particles and creation of a covalent bond between the amphiphilic agent and the particles. This method is specifically described in U.S. Pat. No. 4,578,266.

Particles to which the amphiphilic agents covalently or ionically bond are preferably used.

The amphiphilic agents may represent from 0.1% to 50% by weight, preferably from 0.5% to 30% by weight, and more preferably 1% to 10% by weight, relative to the total weight of the surface-treated particles.

It is preferable that the amphiphilic agent comprises at least one hydrophobicized amino acid.

The hydrophobicized amino acid may be a glutamic acid derivative or a condensate of at least one glutamic acid derivative and an amino acid.

The glutamic acid derivative may be N-acylated glutamic acid or a salt thereof. As the salt, mention may be made of metal salts, ammonium salts, and onium salts of an organic alkanolamine. As the metal, Na, K, Ba, Zn, Ca, Mg, Fe, Zr, Co, Al, and Ti may be used. As the organic alkanolamine, monoethanolamine, diethanolamine, triethanolamine, 2-amino-2-methylpropanol, 2-amino-2-methyl-1,3-propanediol, and triisopropanolamine may be used. The acyl group bound to the nitrogen atom of the glutamic acid may be derived from a saturated or unsaturated fatty acid having 8 to 22 carbon atoms, such as capric acid, lauric acid, myristic acid, isomyristic acid, palmitic acid, isopalmitic acid, stearic acid, isostearic acid, arachic acid, undecylenic acid, oleic acid, myristic acid, elaidic acid, linolic acid, linoleic acid, arachidonic acid, palm oil fatty acid, beef tallow fatty acid, and resin acid (abietic acid).

The condensate of at least one glutamic acid derivative and an amino acid may be a condensate of N-acylated glutamic acid and an amino acid such as lysine, or a salt thereof. As the salt, mention may be made of metal salts, ammonium salts and onium salts of an organic alkanolamine as mentioned above. Sodium salt is preferable. The acyl group bound to the nitrogen atom of the glutamic acid may be derived from a saturated or unsaturated fatty acid having 8 to 22 carbon atoms as mentioned above. Laurie acid is preferable. Thus, for example, sodium dilauramidoglutamide lysine (Pellicer L-30 marketed by Asahi Kasei Chemicals) is preferable as the above condensate.

The amphiphilic surface treatments of the particles may be chosen from the following treatments:
a PEG-silicone treatment, for instance, the AQ surface treatment sold by LCW;
a lauroyllysine treatment, for instance, the LL surface treatment sold by LCW;
a lauroyllysine dimethicone treatment, for instance, the LL/SI surface treatment sold by LCW;
a disodium stearoyl glutamate treatment, for instance, the NAI surface treatment sold by Miyoshi;
a dimethicone/disodium stearoyl glutamate treatment, for instance, the SA/NAI surface treatment sold by Miyoshi;
a microcrystalline cellulose and carboxymethylcellulose treatment, for instance, the AC surface treatment sold by Daito;
an acrylate copolymer treatment, for instance, the APD surface treatment sold by Daito;
a sodium dilauramidoglutamide lysine treatment, for instance, the ASL treatment sold by Daito; and
a sodium dilauramidoglutamide lysine/isopropyl titanium triisostearate treatment, for instance, the ASL treatment sold by Daito.

Amphiphilic agent(s) can be bound to particles ionically with a metal salt or hydroxide whose metal can be selected from Mg, Al, Ca, and Zn, for instance, aluminum hydroxide and magnesium chloride.

A treatment by disodium stearoyl glutamate (and) aluminum hydroxide is more preferable.

Other treatments with a sodium dilauramidoglutamide lysine, or a sodium dilauramidoglutamide lysine/isopropyl titanium triisostearate, are also more preferable.

Preferably uncolored or white water insoluble particles are used.

It is preferable that water insoluble particles are inorganic material s selected from metal oxides, more preferably aluminum or transition metal oxides, and silicon oxides, these compounds being eventually surface treated, and their mixtures.

The amount of the water-insoluble particle(s) in the cosmetic composition is not limited but may be from 0.01 to 30% by weight, preferably from 0.1 to 20% by weight, and more preferably from 0.5 to 10% by weight, relative to the total weight of the composition.

### (4) Water-Soluble Salt

The cosmetic composition according to the present invention comprises at least one water-soluble salt. For the purposes of the present invention, the term “water-soluble salt” means a salt with a solubility in water at 25° C. of 1 wt % or more, preferably forming at this concentration a macroscopically homogeneous, transparent and isotropic medium.

According to the present invention, two or more water-soluble salts may be used in combination. Thus, a single type of water-soluble salt or a combination of different types of water-soluble salts may be used.

The water-soluble salt may be inorganic or organic. The inorganic or organic water-soluble salts that may be used in the present invention can be chosen from water-soluble salts of monovalent or divalent metals, for example, of alkali metal or alkaline-earth metals, of ammonium or of amines, and of mineral acids or of organic carboxylic acids whose anion contains from 1 to 7 carbon atoms. They preferably have a molar mass of between 25 and 650 g/mol.

It is preferable that the water-soluble salt is selected from water-soluble inorganic salts, preferably from silicates, metasilicates, carbonates, hydrogenocarbonates, phosphates, sulfates of alkaline metals, halides of ammonium, halides of alkaline metals, and their mixtures. Examples of such salts that may especially be mentioned include sodium chloride, potassium chloride, calcium chloride, magnesium chloride, ammonium chloride, sodium citrate, ammonium citrate, magnesium sulfate, sodium silicate, sodium metasilicate, sodium carbonate, and the sodium salts of phosphoric acid.

It is more preferable that the water-soluble salt is selected from water-soluble alkaline salts, further more preferably from silicates, metasilicates of alkaline metals, and their mixtures.

Alkaline metal metasilicate such as sodium metasilicate is most preferable.

The amount of the water-soluble salt(s) in the cosmetic composition may be from 0.01 to 50% by weight, preferably from 0.1 to 40% by weight, and more preferably from 0.5 to 30% by weight, relative to the total weight of the composition.

### (5) Alkaline Agent

The cosmetic composition according to the present invention comprises at least one alkaline agent, and two or more alkaline agents may be used. Thus, a single type of alkaline agent or a combination of different types of alkaline agents may be used.

According to a first embodiment, the agent is an inorganic alkaline agent. The inorganic alkaline agent may be aqueous ammonia but is preferably a non-volatile alkaline agent. It is preferable that the inorganic alkaline agent be selected from the group consisting of alkaline metal hydroxides; and alkaline earth metal hydroxides; alkaline metal (hydrogeno)carbonates; alkaline earth metal(hydrogeno)carbonates; and alkaline metal (meta)silicates.

As examples of the inorganic alkaline agents, mention may be made of sodium hydroxide, potassium hydroxide, calcium hydroxide, magnesium hydroxide, sodium carbonate, potassium carbonate, sodium hydrogenocarbonate, potassium hydrogenocarbonate, and sodium (meta)silicate.

According to a second embodiment, the alkaline agent is chosen from organic alkaline agents. It is preferable that these organic alkaline agents be selected from the group of non volatile alkaline agent consisting of monoamines and derivatives thereof, such as alkanolamines; diamines and derivatives thereof, such as alkanoldiamines; polyamines and derivatives thereof; amino acids, preferably basic amino acids, and derivatives thereof; oligomers of amino acids, preferably basic amino acids and derivatives thereof; polymers of amino acids, preferably basic amino acids and derivatives thereof; urea and derivatives thereof; and guanidine and derivatives thereof.

As examples of the organic alkaline agents, mention may be made of alkanolamines such as mono-, di- and tri-ethanolamine, comprising 1 to 3 hydroxyalkyl(C₁-C₄) groups. Particularly, alkanolamines may be selected from monoethanolamine, diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, and tris(hydroxymethylamino)methane.

The organic alkaline agents may also be selected from urea, guanidine and their derivatives; aminoacids such as alanine, arginine, ornithine, citrulline, asparagine, carnitine, cysteine, glutamine, glycine, histidine, lysine, isoleucine, leucine, methionine, N-phenylalanine, proline, serine, threonine, tryptophan, tyrosine and valine, particularly basic amino acids such as lysine, histidine, ornithine, citrulline or arginine; and diamines such as those described in the structure below:

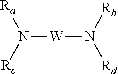

wherein W denotes an alkylene such as propylene optionally substituted by a hydroxyl or a C₁-C₄ alkyl radical, and Rₐ, R_{b}, R_{c} and R_{d} independently denote a hydrogen atom, an alkyl radical or a C₁-C₄ hydroxyalkyl radical, which may be exemplified by 1,3-propanediamine and derivatives thereof. Among aminoacids, basic amino acids such as lysine, histidine, ornithine, citrulline or arginine are preferred.

It is preferable that the alkaline agent be selected from alkanolamines, and particularly monoethanolamine.

It is reminded that the term “organic” means that the alkaline agent has at least one carbon atom in its chemical structure.

It is reminded that the term “non-volatile alkaline agent” means that the alkaline agent has a vapor pressure generally lower than 0.02 mmHg (2.66 Pa) at room temperature.

As previously indicated, the composition comprises at least one water-soluble salt and at least one alkaline agent; these compounds being distinct. Therefore, according to a particular embodiment, the composition may comprise at least two (preferably two) types of alkaline agents, at least one (preferably one) of them being a water-soluble salt.

According to a specific embodiment, the alkaline agent is not a water-soluble salt.

The non-volatile alkaline agent(s) may be used in a total amount of from 0.01 to 15% by weight, preferably from 0.1 to 10% by weight, and more preferably from 1 to 5% by weight, relative to the total weight of the cosmetic composition according to the present invention.

A preferred association of an alkaline agent and a water soluble salt is the association of an alkanolamine, and preferably monoethanolamine, with an alkaline metal metasilicate, preferably sodium metasilicate.

### (6) Surfactant

The cosmetic composition according to the present invention may optionally comprise at least one surfactant in an amount of 1 wt % or less, preferably 0.1 wt % or less, and more preferably 0:01 wt % or less, relative to the total weight of the composition, and most preferably no surfactant.

The surfactant may be selected from the group consisting of anionic surfactants, amphoteric surfactants, cationic surfactants, and nonionic surfactants. Two or more surfactants may be used in combination. Thus, a single type of surfactant or a combination of different types of surfactants may be used. Preferably, according to the present invention, the “surfactant” is capable of forming foam with water without additives.

### (a) Anionic Surfactants

It is preferable that the anionic surfactant be selected from the group consisting of (C₆-C₃₀)alkyl sulfates, (C₆-C₃₀)alkyl ether sulfates, (C₆-C₃₀)alkylamido ether sulfates, alkylaryl polyether sulfates, monoglyceride sulfates; (C₆-C₃₀)alkylsulfonates, (C₆-C₃₀)alkylamide sulfonates, (C₆-C₃₀)alkylaryl sulfonates, α-olefin sulfonates, paraffin sulfonates; (C₆-C₃₀)alkyl phosphates; (C₆-C₃₀)alkyl sulfosuccinates, (C₆-C₃₀)alkyl ether sulfosuccinates, (C₆-C₃₀)alkylamide sulfosuccinates; (C₆-C₃₀)alkyl sulfoacetates; (C₆-C₂₄)acyl sarcosinates; (C₆-C₂₄)acyl glutamates; (C₆-C₃₀)alkylpolyglycoside carboxylic ethers; (C₆-C₃₀)alkylpolyglycoside sulfosuccinates; (C₆-C₃₀)alkyl sulfosuccinamates; (C₆-C₂₄)acyl isethionates; N—(C₆-C₂₄)acyl taurates; C₆-C₃₀ fatty acid salts; coconut oil acid salts or hydrogenated coconut oil acid salts; (C₈-C₂₀)acyl lactylates; (C₆-C₃₀)alkyl-D-galactoside uronic acid salts; polyoxyalkylenated (C₆-C₃₀)alkyl ether carboxylic acid salts; polyoxyalkylenated (C₆-C₃₀)alkylaryl ether carboxylic acid salts; polyoxyalkylenated (C₆-C₃₀)alkylamido ether carboxylic acid salts; and corresponding acids.

It is more preferable that the anionic surfactant be selected from salts of (C₆-C₃₀)alkyl sulfate or polyoxyalkylenated (C₆-C₃₀)alkyl ether carboxylic acid salts.

In at least one embodiment, the anionic surfactants are in the form of salts such as salts of alkali metals, for instance, sodium; salts of alkaline-earth metals, for instance, magnesium; ammonium salts; amine salts; and amino alcohol salts.

### (b) Amphoteric Surfactants

The amphoteric or zwitterionic surfactants can be, for example (nonlimiting list), amine derivatives such as aliphatic secondary or tertiary amine, and optionally quaternized amine derivatives, in which the aliphatic radical is a linear or branched chain comprising 8 to 22 carbon atoms and containing at least one water-solubilizing anionic group (for example, carboxylate, sulphonate, sulphate, phosphate or phosphonate).

The amphoteric surfactant may preferably be selected from the group consisting of betaines and amidoaminecarboxylated derivatives.

The betaine-type amphoteric surfactant is preferably selected from the group consisting of alkylbetaines, alkylamidoalkylbetaines, sulfobetaines, phosphobetaines, and alkylamidoalkylsulfobetaines, in particular, (C₈-C₂₄)alkylbetaines, (C₈-C₂₄)alkylamido(C₁-C₈)alkylbetaines, sulphobetaines, and (C₈-C₂₄)alkylamido(C₁-C₈)alkylsulphobetaines. In one embodiment, the amphoteric surfactants of betaine type are chosen from (C₈-C₂₄)alkylbetaines, (C₈-C₂₄)alkylamido(C₁-C₈)alkylsulphobetaines, sulphobetaines, and phosphobetaines.

Non-limiting examples that may be mentioned include the compounds classified in the CTFA dictionary, 9th edition, 2002, under the names cocobetaine, laurylbetaine, cetylbetaine, coco/oleamidopropylbetaine, cocamidopropylbetaine, palmitamidopropylbetaine, stearamidopropylbetaine, cocamidoethylbetaine, cocamidopropylhydroxysultaine, oleamidopropylhydroxysultaine, cocohydroxysultaine, laurylhydroxysultaine, and cocosultaine, alone or as mixtures.

The betaine-type amphoteric surfactant is preferably an alkylbetaine and an alkylamidoalkylbetaine, in particular, cocobetaine and cocamidopropylbetaine.

Among the amidoaminecarboxylated derivatives, mention may be made of the products sold under the name Miranol, as described in U.S. Pat. Nos. 2,528,378 and 2,781,354 and classified in the CTFA dictionary, 3rd edition, 1982 (the disclosures of which are incorporated herein by reference), under the names Amphocarboxyglycinates and Amphocarboxypropionates, with the respective structures:

R₁—CONHCH₂CH₂—N⁺(R₂)(R₃)(CH₂COO⁻)

in which:
R₁ denotes an alkyl radical of an acid R₁—COOH present in hydrolysed coconut oil, a heptyl, nonyl or undecyl radical,
R₂ denotes a beta-hydroxyethyl group, and
R₃ denotes a carboxymethyl group; and

R₁′—CONHCH₂CH₂—N(B)(C)

in which:
B represents —CH₂CH₂OX′,
C represents —(CH₂)_{z}—Y′, with z=1 or 2,
X denotes a —CH₂CH₂—COOH group, —CH₂—COOZ′, —CH₂CH₂—COOH, —CH₂CH₂—COOZ′ or a hydrogen atom,
Y′ denotes —COOH, —COOZ′, —CH₂—CHOH—SO₃Z′ or a —CH₂—CHOH—SO₃H radical,
Z′ represents an ion of an alkaline or alkaline earth metal such as sodium, an ammonium ion or an ion issued from an organic amine, and
R₁′ denotes an alkyl radical of an acid R₁′—COOH present in coconut oil or in hydrolysed linseed oil, an alkyl radical, such as a C₇, C₉, C₁₁ or C₁₃ alkyl radical, a C₁₇ alkyl radical and its iso form, or an unsaturated C₁₇ radical.

It is preferable that the amphoteric surfactant be selected from (C₈-C₂₄)-alkyl amphomonoacetates, (C₈-C₂₄)alkyl amphodiacetates, (C₈-C₂₄)alkyl amphomonopropionates, and (C₈-C₂₄)alkyl amphodipropionates

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium. Cocoamphodipropionate, Disodium Lauroamphopropionate, Disodium Caprylamphodipropionate, Disodium Caprylamphodipropionate, Lauroamphodipropionic acid, and Cocoamphodipropionic acid.

By way of example, mention may be made of the cocoamphodiacetate sold under the trade name Miranol® C2M concentrate by the company Rhodia Chimie.

### (c) Cationic Surfactants

The cationic surfactant may be selected from the group consisting of optionally polyoxyalkylenated, primary, secondary. or tertiary fatty amine salts, quaternary ammonium salts, and mixtures thereof.

Examples of quaternary ammonium salts that may be mentioned include, but are not limited tothose of general formula (I) below:

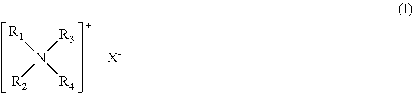

wherein
R₁, R₂, R₃, and R₄, which may be identical or different, are chosen from linear and branched aliphatic radicals comprising from 1 to 30 carbon atoms and optionally comprising heteroatoms such as oxygen, nitrogen, sulfur, and halogens. The aliphatic radicals may be chosen, for example, from alkyl, alkoxy, C₂-C₆ polyoxyalkylene, alkylamide, (C₁₂-C₂₂)alkylamido(C₂-C₆)alkyl, (C₁₂-C₂₂)alkylacetate, and hydroxyalkyl radicals; and aromatic radicals such as aryl and alkylaryl; and X is chosen from halides, phosphates, acetates, lactates, (C₂-C₆) alkyl sulfates, and alkyl- or alkylaryl-sulfonates;
quaternary ammonium salts of imidazoline, for instance, those of formula (II) below:

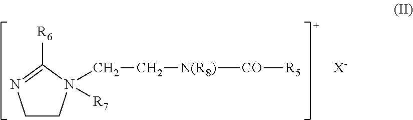

wherein:
R₅ is chosen from alkenyl and alkyl radicals comprising from 8 to 30 carbon atoms, for example fatty acid derivatives of tallow or of coconut;
R₆ is chosen from hydrogen, C₁-C₄ alkyl radicals, and alkenyl and alkyl radicals comprising from 8 to 30 carbon atoms;
R₇ is chosen from C₁-C₄ alkyl radicals;
R₈ is chosen from hydrogen and C₁-C₄ alkyl radicals; and
X⁻is chosen from halides, phosphates, acetates, lactates, alkyl sulfates, alkyl sulfonates, and alkylaryl sulfonates. In one embodiment, R₅ and R₆ are, for example, a mixture of radicals chosen from alkenyl and alkyl radicals comprising from 12 to 21 carbon atoms, such as fatty acid derivatives of tallow, R₇ is methyl and R₈ is hydrogen. Examples of such products include, but are not limited to, Quaternium-27 (CTFA 1997) and Quaternium-83 (CTFA 1997), which are sold under the names “Rewoquat®” W75, W90, W75PG and W75HPG by the company Witco; diquaternary ammonium salts of formula (III):

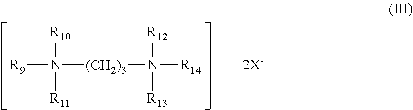

wherein:
R₉ is chosen from aliphatic radicals comprising from 16 to 30 carbon atoms;
R₁₀ is chosen from hydrogen or alkyl radicals comprising from 1 to 4 carbon atoms or the group (R₁₆ₐ)(R₁₇ₐ)(R₁₈ₐ)N⁺(CH₂)₃;
R₁₁, R₁₂, R₁₃, R₁₄, R₁₆ₐ, R₁₇ₐ, and R₁₈ₐ, which may be identical or different, are chosen from hydrogen and alkyl radicals comprising from 1 to 4 carbon atoms; and

X⁻is chosen from halides, acetates, phosphates, nitrates, ethyl sulfates, and methyl sulfates.

An example of one such diquaternary ammonium salt is FINQUAT CTP of FINETEX(Quaternium-89) or FINQUAT CT of FINETEX (Quatemium-75); and quaternary ammonium salts comprising at least one ester function, such as those of formula (IV) below:

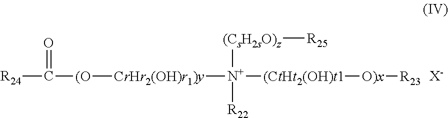

wherein:
R₂₂ is chosen from C₁-C₆ alkyl radicals and C₁-C₆ hydroxyalkyl and dihydroxyalkyl radicals;
R₂₃ is chosen from:
the radical below:

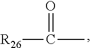

linear and branched, saturated and unsaturated C₁-C₂₂ hydrocarbon-based radicals R₂₇, and hydrogen,
R₂₅ is chosen from:
the radical below:

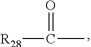

linear and branched, saturated and unsaturated C₁-C₆ hydrocarbon-based radicals R₂₉, and hydrogen,
R₂₄, R₂₆, and R₂₈, which may be identical or different, are chosen from linear and branched, saturated and unsaturated, C₇-C₂₁, hydrocarbon-based radicals;
r, s, and t, which may be identical or different, are chosen from integers ranging from 2 to 6; each of r1 and t1, which may be identical or different, is 0 or 1, and r2+r1=2r and t1+2t=2t;
y is chosen from integers ranging from 1 to 10;
x and z, which may be identical or different, are chosen from integers ranging from 0 to 10;
X⁻ is chosen from simple and complex, organic and inorganic anions; with the proviso that the sum x+y+z ranges from 1 to 15, that when x is 0, R₂₃ denotes R₂₇, and that when z is 0, R₂₅ denotes R₂₉. R₂₂ may be chosen from linear and branched alkyl radicals. In one embodiment, R₂₂ is chosen from linear alkyl radicals. In another embodiment, R₂₂ is chosen from methyl, ethyl, hydroxyethyl, and dihydroxypropyl radicals, for example, methyl and ethyl radicals. In one embodiment, the sum x+y+z ranges from 1 to 10. When R₂₃ is a hydrocarbon-based radical R₂₇, it may be long and comprise from 12 to 22 carbon atoms, or short and comprise from 1 to 3 carbon atoms. When R₂₅ is a hydrocarbon-based radical R₂₉, it may comprise, for example, from 1 to 3 carbon atoms. By way of a non-limiting example, in one embodiment, R₂₄, R₂₆, and R₂₈, which may be identical or different, are chosen from linear and branched, saturated and unsaturated, C₁₁-C₂₁ hydrocarbon-based radicals, for example, from linear and branched, saturated and unsaturated C₁₁-C₂₁ alkyl and alkenyl radicals. In another embodiment, x and z, which may be identical or different, are 0 or 1. In one embodiment, y is equal to 1. In another embodiment, r, s, and t, which may be identical or different, are equal to 2 or 3, for example, equal to 2. The anion X⁻ may be chosen from, for example, halides, such as chloride, bromide, and iodide; and C₁-C₄ alkyl sulfates, such as methyl sulfate. However, methanesulfonate, phosphate, nitrate, tosylate, an anion derived from an organic acid, such as acetate and lactate, and any other anion that is compatible with the ammonium comprising an ester function, are other non-limiting examples of anions that may be used according to the invention. In one embodiment, the anion X⁻ is chosen from chloride and methyl sulfate.

In another embodiment, the ammonium salts of formula (IV) may be used, wherein:

R₂₂ is chosen from methyl and ethyl radicals,
x and y are equal to 1;
z is equal to 0 or 1;
r, s, and t are equal to 2;
R₂₃ is chosen from:
the radical below:

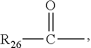

methyl, ethyl, and C₁₄-C₂₂ hydrocarbon-based radicals, and hydrogen;
R₂₅ is chosen from:
the radical below:

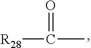

and hydrogen;
R₂₄, R₂₆, and R₂₈, which may be identical or different, are chosen from linear and branched, saturated and unsaturated, C₁₃-C₁₇ hydrocarbon-based radicals, for example, from linear and branched, saturated and unsaturated, C₁₃-C₁₇ alkyl and alkenyl radicals.

In one embodiment, the hydrocarbon-based radicals are linear.

Non-limiting examples of compounds of formula (IV) that may be mentioned include salts, for example chloride and methyl sulfate, of diacyloxyethyl-dimethylammonium, of diacyloxyethyl-hydroxyethyl-methylammonium, of monoacyloxyethyl-dihydroxyethyl-methylammonium, of triacyloxyethyl-methylammonium, of monoacyloxyethyl-hydroxyethyl-dimethyl-ammonium, and mixtures thereof. In one embodiment, the acyl radicals may comprise from 14 to 18 carbon atoms, and may be derived, for example, from a plant oil, for instance palm oil and sunflower oil. When the compound comprises several acyl radicals, these radicals may be identical or different.

These products may be obtained, for example, by direct esterification of optionally oxyalkylenated triethanolamine, triisopropanolamine, alkyldiethanolamine, or alkyldiisopropanolamine onto fatty acids or onto mixtures of fatty acids of plant or animal origin, or by transesterification of the methyl esters thereof. This esterification may be followed by a quaternization using an alkylating agent chosen from alkyl halides, for example, methyl and ethyl halides; dialkyl sulfates, for example, dimethyl and diethyl sulfates; methyl methanesulfonate; methyl para-toluenesulfonate; glycol chlorohydrin; and glycerol chlorohydrin.

Such compounds are sold, for example, under the names Dehyquart® by the company Cognis, Stepanquat® by the company Stepan, Noxamium® by the company Ceca, and “Rewoquat® WE 18” by the company Rewo-Goldschmidt.

Other non-limiting examples of ammonium salts that may be used in the compositions according to the invention include the ammonium salts comprising at least one ester function described in U.S. Pat. Nos. 4,874,554 and 4,137,180.

The quaternary ammonium salts mentioned above that may be used in compositions according to the present invention include, but are not limited to, those corresponding to formula (I), for example tetraalkylammonium chlorides, for instance, dialkyldimethylammonium and alkyltrimethylammonium chlorides in which the alkyl radical comprises from about 12 to 22 carbon atoms, such as behenyltrimethylammonium, distearyldimethylammonium, cetyltrimethylammonium, and benzyldimethylstearylammonium chloride; palmitylamidopropyltrimethylammonium chloride; and stearamidopropyldimethyl(myristyl acetate)ammonium chloride, sold under the name “Ceraphyl® 70” by the company Van Dyk.

According to one embodiment, the cationic surfactant that may be used in the compositions of the present invention is chosen from quaternary ammonium salts, for example, from behenyltrimethylammonium chloride, cetyltrimethylammonium chloride, Quatemium-83, Quatemium-87, Quaternium-22, behenylamidopropyl-2,3-dihydroxypropyldimethylammonium chloride, palmitylamidopropyltrimethylammonium chloride, and stearamidopropyldimethylamine.

### (d) Nonionic Surfactants

The nonionic surfactants are compounds well known in themselves (in this regard, see, for example, “Handbook of Surfactants” by M. R. Porter, Blackie & Son publishers (Glasgow and London), 1991, pp. 116-178). Thus, they can, for example, be chosen from alcohols, alpha-diols, alkylphenols and esters of fatty acids that are polyethoxylated, polypropoxylated, or polyglycerolated and have at least one fatty chain comprising, for example, from 8 to 18 carbon atoms, it being possible for the number of ethylene oxide or propylene oxide groups to range from 2 to 50, and for the number of glycerol groups to range from 2 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1 to 5 glycerol groups, such as from 1.5 to 4; ethoxylated fatty acid esters of sorbitan comprising from 2 to 30 mol of ethylene oxide; ethoxylated oils of plant origin; fatty acid esters of sucrose; fatty acid esters of polyethylene glycol; fatty acid mono or diesters of glycerol; (C₆-C₂₄)alkylpolyglycosides; N—(C₆-C₂₄)alkylglucamine derivatives, amine oxides such as (C₁₀-C₁₄)alkylamine oxides, or N—(C₁₀-C₁₄)acylaminopropylmorpholine oxides; and mixtures thereof.

The nonionic surfactants may preferably be chosen from monooxyalkylenated or polyoxyalkylenated, monoglycerolated, or polyglycerolated nonionic surfactants. The oxyalkylene units are more particularly oxyethylene or oxypropylene units, or a combination thereof, preferably oxyethylene units.

Examples of oxyalkylenated nonionic surfactants that may be mentioned include: oxyalkylenated (C₈-C₂₄)alkylphenols,

saturated or unsaturated, linear or branched, oxyalkylenated C₈-C₃₀ alcohols,
saturated or unsaturated, linear or branched, oxyalkylenated C₈-C₃₀ amides, esters of saturated or unsaturated, linear or branched, C₈-C₃₀ acids and of polyethylene glycols, polyoxyalkylenated esters of saturated or unsaturated, linear or branched, C₈-C₃₀ acids and of sorbitol,
saturated or unsaturated, oxyalkylenated plant oils,
condensates of ethylene oxide and/or of propylene oxide, inter alia, alone or as mixtures.

The surfactants may contain a number of moles of ethylene oxide and/or of propylene oxide of between 1 and 100 and preferably between 2 and 50. Advantageously, the nonionic surfactants do not comprise any oxypropylene units.

In accordance with one preferred embodiment of the invention, the oxyalkylenated nonionic surfactants are chosen from oxyethylenated C₈-C₃₀ alcohols or ethoxylated fatty esters.

Examples of ethoxylated fatty alcohols (or C₈-C₃₀ alcohols) that may be mentioned include the adducts of ethylene oxide with lauryl alcohol, especially those containing from 9 to 50 oxyethylene groups and more particularly those containing from 10 to 12 oxyethylene groups (Laureth-10 to Laureth-12, as the CTFA names); the adducts of ethylene oxide with behenyl alcohol, especially those containing from 9 to 50 oxyethylene groups (Beheneth-9 to Beheneth-50, as the CTFA names); the adducts of ethylene oxide with cetearyl alcohol (mixture of cetyl alcohol and stearyl alcohol), especially those containing from 10 to 30 oxyethylene groups (Ceteareth-10 to Ceteareth-30, as the CTFA names); the adducts of ethylene oxide with cetyl alcohol, especially those containing from 10 to 30 oxyethylene groups (Ceteth-10 to Ceteth-30, as the CTFA names); the adducts of ethylene oxide with stearyl alcohol, especially those containing from 10 to 30 oxyethylene groups (Steareth-10 to Steareth-30, as the CTFA names); the adducts of ethylene oxide with isostearyl alcohol, especially those containing from 10 to 50 oxyethylene groups (Isosteareth-10 to Isosteareth-50, as the CTFA names); and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 50 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); and mixtures thereof.

Mixtures of these oxyethylenated derivatives of fatty alcohols and of fatty esters may also be used.

According to one preferred embodiment of the invention, the cosmetic composition according to the present invention comprises at least one ethoxylated fatty alcohol.

As examples of monoglycerolated or polyglycerolated nonionic surfactants, monoglycerolated or polyglycerolated C₈-C₄₀ alcohols are preferably used.

In particular, the monoglycerolated or polyglycerolated C₈-C₄₀ alcohols correspond to the following formula:

RO—[CH₂—CH(CH₂OH)—O]ₘ—H or RO—[CH(CH₂OH)—CH₂O]ₘ—H

in which R represents a linear or branched C₈-C₄₀ and preferably C₈-C₃₀ alkyl or alkenyl radical, and m represents a number ranging from 1 to 30 and preferably from 1 to 10.

As examples of compounds that are suitable in the context of the invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

Among the monoglycerolated or polyglycerolated alcohols, it is more particularly preferred to use the C₈/C₁₀ alcohol containing 1 mol of glycerol, the C₁₀/C₁₂ alcohol containing 1 mol of glycerol, and the C₁₂ alcohol containing 1.5 mol of glycerol.

Preferably, the nonionic surfactant may be a nonionic surfactant with an HLB from 8 to 18. The HLB is the ratio between the hydrophilic part and the lipophilic part in the molecule. This term HLB is well known to those skilled in the art and is described in “The HLB system. A time-saving guide to emulsifier selection” (published by ICI Americas Inc., 1984).

### (7) Thickening Agent

The cosmetic composition according to the present invention can comprise at least one thickening agent.

According to the present invention, two or more thickening agents may be used in combination.

Thus, a single type of thickening agent or a combination of different types of thickening agents may be used.

The thickening agent may be a rheology modifier selected from hydrophilic or lipophilic, organic or inorganic polymers and non-polymers. Preferably, thickening agents are organic polymers.

The rheology modifier may preferably be chosen from cellulose polymers, galactomannans and derivatives thereof, gums of microbial origin, acrylic acid or acrylamidopropanesulfonic acid crosslinked homopolymers; associative polymers; and mixtures thereof.

As the cellulose polymers, for example, mention may be made of: hydroxyethylcelluloses, hydroxypropylcelluloses, methylcelluloses, ethylhydroxyethylcelluloses, carboxymethylcelluloses, and quaternized cellulose derivatives.

As microbial gums, mention may be made of xanthan gum and scieroglucan gum.

As the acrylic acid or acrylamidopropanesulfonic acid crosslinked homopolymers, for example, mention may be made of:

acrylic acid homopolymers crosslinked with an allylic alcohol ether of the sugar series, for example, the products sold under the names CARBOPOL 980, 981, 954, 2984, and 5984 by the company Goodrich and the products sold under the names SYNTHALEN M and SYNTHALEN K by the company 3 VSA;
crosslinked acrylamidomethanesulfonic acid homopolymers, crosslinked acrylamidoethanesulfonic acid homopolymers, crosslinked acrylamidopropanesulfonic acid homopolymers, crosslinked 2-acrylamido-2-methylpropanesulfonic acid homopolymers, crosslinked 2-methylacrylamido-2-methylpropanesulfonic acid homopolymers, and crosslinked 2-acrylamido-n-butanesulfonic acid homopoplymers, in particular, crosslinked and partially or totally neutralized poly-2-acrylamido-2-methylpropanesulfonic acids are described and prepared, for example, in German Patent No. 196 25 810.

As the associative polymer, any amphiphilic polymer comprising, in its structure, at least one fatty chain and at least one hydrophilic portion, may be used.

The associative polymers in accordance with the present disclosure may be chosen from anionic, cationic, nonionic, and amphoteric polymers.

Among the associative anionic polymers that non-limiting mention may be made of, those comprising at least one hydrophilic unit and at least one fatty-chain allyl ether unit, for example, those in which the at least one hydrophilic unit comprises at least one ethylenic unsaturated anionic monomeric residue chosen, for example, from the residue of a vinylcarboxylic acid, the residue of an acrylic acid, and the residue of a methacrylic acid, and in which the at least one fatty-chain allyl ether unit is chosen from the residues of the monomers of the formula below:

CH₂═(R′)CH₂OBₙR

wherein R is chosen from H and CH₃, B is an ethylenoxy radical, n is 0 or an integer ranging from 1 to 100, and R is chosen from hydrocarbon-based radicals chosen from alkyl, arylalkyl, aryl, alkylarylm and cycloalkyl radicals comprising from 8 to 30 carbon atoms, for example, from 10 to 24 and further, for example, from 12 to 18 carbon atoms.

Anionic amphiphilic polymers of this type are described and prepared, for example, according to an emulsion polymerization process described in document EP-0 216 479.

Associative anionic polymers that non-limiting mention may be made of include anionic polymers comprising at least one hydrophilic unit of olefinic unsaturated carboxylic acid type, and at least one hydrophobic unit exclusively of (C₁₀-C₃₀) alkyl ester of unsaturated carboxylic acid type.

Further examples include the anionic polymers described and prepared according to U.S. Pat. Nos. 3,915,921 and 4,509,949.

Cationic associative polymers that non-limiting mention may be made of include quaternized cellulose derivatives and polyacrylates comprising at least one amine side group.

The nonionic associative polymers may be chosen from at least one of:

celluloses modified with groups comprising at least one fatty chain, for example, hydroxyethylcelluloses modified with groups comprising at least one fatty chain, such as alkyl groups, for example, C₈-C₂, arylalkyl and alkylaryl groups, for example, Natrosol Plus Grade 330 CS (C₁₆ alkyls) sold by the company Aqualon,
celluloses modified with polyalkylene glycol alkylphenyl ether groups,
guars, for example, hydroxypropyl guar, modified with groups comprising at least one fatty chain, for example, an alkyl chain,
copolymers of vinylpyrrolidone and of fatty-chain hydrophobic monomeric residues, copolymers of at least one monomeric residue chosen from C₁-C₆ alkyl methacrylates and acrylates, and of amphiphilic monomeric residues comprising at least one fatty chain, copolymers of monomeric residues chosen from hydrophilic methacrylates and acrylates, and of hydrophobic monomeric residues comprising at least one fatty chain, for example, the polyethylene glycol methacrylate/lauryl methacrylate copolymer,
associative polyurethanes, and
mixtures thereof.

For example, the associative polymers may be chosen from associative polyurethanes.

In another example, associative polyurethanes may be chosen from nonionic block copolymers comprising in the chain both hydrophilic blocks usually of polyoxyethylene nature, and hydrophobic blocks that may be chosen from aliphatic sequences, cycloaliphatic sequences, and aromatic sequences.

Further, for example, these polymers may comprise at least two hydrocarbon-based lipophilic chains comprising from 6 to 30 carbon atoms, separated by a hydrophilic block, wherein the hydrocarbon-based lipophilic chains may be chosen from pendant chains and chains at the end of a hydrophilic block. In yet another example, the polymers may comprise at least one pendant chain. In another example, the polymers may comprise a hydrocarbon-based chain at one or both ends of a hydrophilic block.

For example, the associative polyurethanes may be blocked in triblock or multiblock form. The hydrophobic blocks may thus be at each end of the chain (for example, triblock copolymer with a hydrophilic central block) or distributed both at the ends and within the chain (for example multiblock copolymer). These polymers may also be chosen from graft polymers and starburst polymers.

In another example, the associative polyurethanes are triblock copolymers in which the hydrophilic block is a polyoxyethylene chain comprising from 50 to 1000 oxyethylene groups. In general, the associative polyurethanes comprise a urethane bond between the hydrophilic blocks, hence the name.

By way of example, among the associative polymers that may be used, non-limiting mention may be made of the polymer C₁₆-OE₁₂₀-C₁₆ from the company Servo Delden (under the name SER AD FX1100, which comprises a urethane functional group and has a weight-average molecular weight of 1300), wherein OE is an oxyethylene unit. In another example, an associative polymer that may also be used is Rheolate 205 comprising a urea functional group, sold by the company Rheox, or Rheolate 208 or 204. These associative polyurethanes are sold in pure form.

In yet another example, the product DW 1206B from Rohm & Haas comprising a C₂₀ alkyl chain with a urethane bond, sold at a solids content of 20% in water, may also be used.

Further, for example, it is also possible to use solutions or dispersions of these polymers, for example, in water or in aqueous-alcoholic medium. Examples of such polymers that non-limiting mention may be made of include SER AD FX1010, SER AD FX1035 and SER AD 1070 from the company Servo Delden, and Rheolate 255, Rheolate 278, and Rheolate 244 sold by the company Rheox. It is also possible to use the product DW 1206F and DW 1206J, and also Acrysol RM 184 or Acrysol 44 from the company Rohm & Haas, or alternatively Borchigel LW 44 from the company Borchers.

In yet another example, the polymers that may be used include those described in the article by G Fonnum, J. Bakke and Fk. Hansen—Colloid Polym. Sci 271, 380-389 (1993).

The thickening agent may more preferably be selected from cellulose polymers or microbial gums.

According to one embodiment of the present invention, the amount of the thickening agent(s), preferably organic thickening polymers, may range from 0.05 to 20% by weight, preferably from 0.1 to 15% by weight, and more preferably from 0.5 to 10% by weight, relative to the total weight of the cosmetic composition according to the present invention.

### (8) Coloring Substance

The cosmetic composition according to the present invention can comprise at least one dyeing substance which is different from pigments. Thus, a single type of dyeing substance or a combination of different types of coloring substances may be used.

The dyeing substance may be an oxidation dye.

The oxidation dye can be selected from oxidation bases, oxidation couplers, and the acid addition salts thereof.

The oxidation base can be selected from those conventionally known in oxidation dyeing, preferably from the group consisting of ortho- and para-phenylenediamines, double bases, ortho- and para-aminophenols, heterocyclic bases, and the acid addition salts thereof.

Among the para-phenylenediamines, may be mentioned more particularly para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethylpara-phenylenediamine, N,N-diethyl-para-phenylenediamine, N,N-dipropyl-paraphenylenediamine, 4-amino-N,N-diethyl-3-methylaniline, N,N-bis((β-hydroxyethyl)-paraphenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-paraphenylenediamine, 2-isopropyl-para-phenylenediamine, N-(β-hydroxypropyl)-paraphenylenediamine, 2-hydroxymethyl-para-phenylenediamine, N,N-dimethyl-3-methylpara-phenylenediamine, N,N-(ethyl-β-hydroxyethyl)-para-phenylenediamine, N-β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylamino-ethyloxy-para-phenylenediamine, N-(β-methoxyethyl)-para-phenylenediamine, 2-methyl-1-N-β-hydroxyethyl-para-phenylenediamine, N-(4-aminophenyl)-3-hydroxy-pyrrolidine, 2-[{2-[(4-Aminophenyl)amino]ethyl}(2-hydroxyéthyl)amino]-ethanol, and addition salts thereof with an acid. Most particularly preferred bases are para-phenylenediamine, para-tolylenediamine, 2-isopropyl-paraphenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, and addition salts thereof with an acid.

Among the double bases, the following bases can be cited N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylene-diamine, 1,8-bis(2,5-diaminophenoxy)-3,5-dioxaoctane, and addition salts thereof with an acid.

The para-aminophenols that can be used are para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, and addition salts thereof with an acid.

The ortho-aminophenols which can be used as oxidation bases in the context of the present invention are chosen in particular from 2-aminophenol, 2-amino-1-hydroxy-5-methylbenzene, 2-amino-1-hydroxy-6-methylbenzene, 5-acetamido-2-aminophenol, and addition salts thereof with an acid.

Among the heterocyclic bases which can be used as oxidation bases in the dyeing compositions in accordance with the present invention, there may be mentioned more particularly pyridine derivatives, pyrimidine derivatives, pyrazole derivatives, and addition salts thereof with an acid.

Among the pyridine derivatives, there may be mentioned more particularly the compounds described for example in patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and addition salts thereof with an acid.

Among the pyrimidine derivatives, there may be mentioned more particularly the compounds described, for example, in patents DE 2 359 399, JP 88-169571, and JP 91-10659. or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triamino-pyrimidine, and the pyrazolopyrimidine derivatives such as those mentioned in patent application FR-A-2 750 048 and among which there may be mentioned pyrazolo[1,5-a]-pyrimidine-3,7-diamine; 2,5-dimethyl-pyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-amino-pyrazolo[1,5-a]pyrimidin-5-ol; 2-(3-amino-pyrazolo[1,5-a]pyrimidin-7-ylamino)ethanol, 2-(7-aminopyrazolo[1,5-a]pyrimidin-3-ylamino)ethanol, 2-[(3-amino-pyrazolo[1,5-a]pyrimidin-7-yl)-(2-hydroxy-ethyl)amino]-ethanol, 2-[(7-aminopyrazolo[1,5-a]-pyrimidin-3-yl)-(2-hydroxyethyl)amino]ethanol, 5,6-dimethylpyrazolo-[1,5-a]pyrimidine-3,7-diamine, 2,6-dimethylpyrazolo-[1,5-a]pyrimidine-3,7-diamine, 2,5,N7,N7-tetramethyl-pyrazolo[1,5-a]pyrimidine-3,7-diamine, 3-amino-5-methyl-7-imidazolylpropyl-aminopyrazolo[1,5-a]-pyrimidine, addition salts thereof and their tautomeric forms, when a tautomeric equilibrium exists, and addition salts thereof with an acid.

Among the pyrazole derivatives, there may be mentioned more particularly the compounds described in patents DE 3 843 892 and DE 4 133 957 and patent applications WO 94/08969, WO 94/08970, FR-A-2 733 749, and DE 195 43 988, such as 4,5-diamino-1-methylpyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazino-pyrazole, 1-benzyl-4,5-diamino-3-methyl-pyrazole, 4,5-diamino-3-tert-butyl-1-methylpyrazole, 4,5-diamino-1-tertbutyl-3-methylpyrazole, 4,5-diamino-1-(β-hydroxyethyl)-3-methylpyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxy-methylpyrazole, 4,5-diamino-3-hydroxymethyl-1-methylpyrazole, 4,5-diamino-3-hydroxymethyl-1-isopropyl-pyrazole, 4,5-diamino-3-methyl-1-isopropyl-pyrazole, 4-amino-5-(2′-aminoethyl)amino-1,3-dimethylpyrazole, 3,4,5-triaminopyrazole, 1-methyl-3,4,5-triamino-pyrazole, 3,5-diamino-1-methyl-4-methylaminopyrazole, 3,5-diamino-4-(β-hydroxy-ethyl)amino-1-methylpyrazole, and addition salts thereof with an acid.

Among the heterocyclic bases which can be used as oxidation bases, there may be mentioned more particularly diaminopyrazolopyrazolones and especially 2,3-diamino-6,7-dihydro-1H,5H-[pyrazolo1,2,a]pyrazol-1-one (IV), and the addition salts of these diaminopyrazolopyrazolones with an acid.

The oxidation dye may be an oxidation coupler which can be selected from those conventionally known in oxidation dyeing, preferably from the group consisting of meta-phenylenediamines, meta-aminophenols, meta-diphenols, naphthols, heterocyclic couplers, and the acid addition salts thereof.

The heterocyclic couplers may be selected from the group consisting of indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines, and addition salts thereof with an acid.

These couplers are more particularly chosen from 2,4-diamino-1-(β-hydroxyethyloxy)benzene, 2-methyl-5-aminophenol, 5-N-(β-hydroxyethyl)amino-2-methylphenol, 3-aminophenol, 2-chloro-3-amino-6-methylphenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 2-methyl-5-hydroxyethylaminophenol, 4-amino-2-hydroxytoluene, 1,3-bis(2,4-diaminophenoxy)-propane, sesamol, 1-amino-2-methoxy-4,5-methylene-dioxybenzene, α-naphthol, 6-hydroxyindole, 4-hydroxyindole, 4-hydroxy-N-methylindole, 6-hydroxy-indoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethyl-pyrazolo[3,2-c]-1,2,4-triazole, 2,6-dimethylpyrazolo[1,5-b]-1,2,4-triazole, and addition salts thereof with an acid.

In general, the addition acid salts of the oxidation bases and couplers are chosen in particular from hydrochlorides, hydrobromides, sulphates, tartrates, lactates, and acetates.

The cosmetic composition according to the present invention may comprise oxidation dye(s) in an amount of from 0.0001 to 20% by weight, preferably from 0.0005 to 15% by weight, and more preferably from 0.005 to 10% by weight, relative to the total weight of the composition.

The coloring substance may be a direct dye.

The direct dye can be selected from ionic and nonionic species, preferably cationic or nonionic species.

Examples of suitable direct dyes that may be mentioned include the following direct dyes: azo dyes; methine dyes; carbonyl dyes; azine dyes; nitro (hetero)aryl dyes; tri(hetero)arylmethane dyes; porphyrin dyes; phthalocyanin dyes, and natural direct dyes, alone or as mixtures.

More particularly, the azo dyes comprise an —N═N— function, the two nitrogen atoms of which are not simultaneously engaged in a ring. However, it is not excluded for one of the two nitrogen atoms of the sequence —N═N— to be engaged in a ring.

The dyes of the methine family are more particularly compounds comprising at least one sequence chosen from >C═C< and —N═C<, the two atoms of which are not simultaneously engaged in a ring. However, it is pointed out that one of the nitrogen or carbon atoms of the sequences may be engaged in a ring. More particularly, the dyes of this family are derived from compounds of the type such as methines, azomethines, mono- and diarylmethanes, indoamines (or diphenyl-amines), indophenols, indoanilines, carbocyanins, azacarbocyanins and isomers thereof, diazacarbocyanins and isomers thereof, tetraazacarbocyanins and hemicyanins.

As regards the dyes of the carbonyl family, examples that may be mentioned include dyes chosen from acridone, benzoquinone, anthraquinone, naphthoquinone, benzanthrone, anthranthrone, pyranthrone, pyrazolanthrone, pyrimidinoanthrone, flavanthrone, idanthrone, flavone, (iso)violanthrone, isoindolinone, benzimidazolone, isoquinolinone, anthrapyridone, pyrazolo-quinazolone, perinone, quinacridone, quinophthalone, indigoid, thioindigo, naphthalimide, anthrapyrimidine, diketopyrrolopyrrole, and coumarin.

As regards the dyes of the cyclic azine family, mention may be made especially of azine, xanthene, thioxanthene, fluorindine, acridine, (di)oxazine, (di)thiazine, and pyronin.

The nitro (hetero)aromatic dyes are more particularly nitrobenzene or nitropyridine direct dyes.

As regards the dyes of porphyrin or phthalocyanin type, it is possible to use cationic or non-cationic compounds, optionally comprising one or more metals or metal ions, for instance, alkali metals, alkaline-earth metals, zinc, and silicon.

Examples of particularly suitable direct dyes that may be mentioned include nitrobenzene dyes; azo direct dyes; azomethine direct dyes; methine direct dyes; azacarbocyanin direct dyes, for instance, tetraazacarbocyanins (tetraazapentamethines); quinone and in particular anthraquinone, naphthoquinone or benzoquinone direct dyes; azine; xanthene; triarylmethane; indoamine; indigoid; phthalocyanin direct dyes, porphyrins and natural direct dyes, alone or as mixtures thereof.

Among the natural direct dyes that may be used according to the present invention, mention may be made of lawsone, juglone, alizarin, purpurin, carminic acid, kermesic acid, purpurogallin, protocatechaldehyde, indigo, isatin, curcumin, spinulosin, apigenidin, and orceins. It is also possible to use extracts or decoctions containing these natural dyes and especially henna-based poultices or extracts.

When they are present, the direct dyes more particularly represent from 0.0001 to 10% by weight, and preferably from 0.005 to 5% by weight, relative to the total weight of the composition.

### (9) Other Components

The cosmetic composition according to the present invention may also comprise an effective amount of other agents, known previously elsewhere in lightening or coloring compositions, such as various common adjuvants, sequestering agents such as EDTA and etidronic acid, UV screening agents, silicones other than those mentioned before such as organomodified silicones (such as with amine groups); preserving agents, vitamins or provitamins, for instance, panthenol, opacifiers, fragrances, plant extracts, cationic polymers and so on.

The cosmetic composition according to the present invention may comprise an aqueous medium.

The aqueous medium in the cosmetic composition according to the present invention comprises water. The amount of water may be less than 80 wt %, preferably 5 wt % to 75 wt %, more preferably 10 wt % to 75 wt %, and even more preferably 20 wt % to 70 wt %, relative to the total weight of the composition.

The aqueous phase may further comprise at least one organic solvent. So the organic solvent is preferably water miscible. As the organic solvent, there may be mentioned, for example, C₁-C₄ alkanols, such as ethanol and isopropanol; polyols and polyol ethers such as glycerol, 2-butoxyethanol, propylene glycol, monomethyl ether of propylene glycol, monoethyl ether, and monomethyl ether of diethylene glycol; and aromatic alcohols such as benzyl alcohol and phenoxyethanol; analogous products; and mixtures thereof.

The organic water-soluble solvents may be present in an amount ranging from 1 to 40 wt %, preferably from 1 to 30 wt %, and more preferably from 5 to 20 wt %, relative to the total weight of the composition.

The pH of the cosmetic composition according to the present invention may be generally, for example, from 4 to 12. It can range from 6 to 12, preferably 7 to 11, and may be adjusted to the desired value using at least one acidifying agent that is well known in the prior art.

The acidifying agents can be, for example, mineral or organic acids, for instance, hydrochloric acid and orthophosphoric acid, carboxylic acids, for instance, tartaric acid, citric acid, and lactic acid, or sulphonic acids.

The viscosity of the cosmetic composition according to the present invention is not particularly limited. The viscosity can be measured at 25° C. with viscosimeters or rheometers preferably with coneplan geometry. Preferably, the viscosity of the cosmetic composition according to the present invention can range, for example, from 1 to 2000 Pa·s, and preferably from 1 to 1000 Pa·s at 25° C. and 1 s⁻¹.

The composition according to the invention may also comprise a propellant. For the purposes of the present invention, the term “propellant” means any compound that is gaseous at a temperature of 20° C. and at atmospheric pressure, and that can be stored under pressure in liquid or gaseous form in an aerosol container.

The propellant may be chosen from optionally halogenated volatile hydrocarbons, such as n-butane, propane, isobutane, pentane, or a halogenated hydrocarbon, and mixtures thereof. Carbon dioxide, nitrous oxide, dimethyl ether (DME), nitrogen, or compressed air may also be used as the propellant. Mixtures of propellants may also be used. Dimethyl ether and/or non-halogenated volatile hydrocarbons are preferably used.

The propellant may be present in the composition in a content of between 1% and 15%, preferably between 2% and 10%, and more preferably between 3% and 8% by weight, relative to the total weight of the composition.

The cosmetic composition according to the present invention can be prepared by mixing the dispersed phase(s), the continuous phase, the water-insoluble particles, the water-soluble salt(s) and the alkaline agent(s).

In particular, the cosmetic composition according to the present invention can be prepared by mixing at least the essential components (3) to (5), together with the above optional component(s), if necessary, typically component (2) and water.

The present invention also relates to a process of dyeing or enlightening keratin fibers such as hair, comprising applying the cosmetic composition as described above to the keratin fibers, in the presence of an oxidizing agent.

As the oxidizing agent, a single type of oxidizing agent or a combination of different type of oxidizing agents may be used.

The oxidizing agent may be chosen from hydrogen peroxide, peroxygenated salts, and compounds capable of producing hydrogen peroxide by hydrolysis. For example, the oxidizing agent can be chosen from aqueous hydrogen peroxide solution, urea peroxide, alkali metal bromates, and ferricyanides, and persalts such as perborates and persulphates.

It is preferable that the oxidizing agent be hydrogen peroxide.

The oxidizing agent's concentration may range from 0.1 to 15% by weight, preferably from 0.5 to 10% by weight, and more preferably 1 to 5% by weight, relative to the total weight of the ready-to-use cosmetic composition according to the present invention.

In one embodiment, when the oxidizing agent is hydrogen peroxide, the ready-to-use composition may comprise at least one hydrogen peroxide stabilizer, which may be chosen, for example, from alkali metal and alkaline-earth metal pyrophosphates, alkali metal and alkaline-earth metal stannates, phenacetin, and salts of acids and of oxyquinoline, for example, oxyquinoline sulphate. In another embodiment, at least one stannate optionally in combination with at least one pyrophosphate is used.

It is also possible to use salicylic acid and salts thereof, pyridinedicarboxylic acid and its salts, and paracetamol.

Moreover, the concentration of the hydrogen peroxide stabilizer may range from 0.0001 to 5% by weight such as from 0.01 to 2% by weight, relative to the total weight of the ready-to-use composition.

In the composition comprising hydrogen peroxide, the concentration ratio of the hydrogen peroxide to the stabilizer may range from 0.05:1 to 1,000:1, such as from 0.1:1 to 500:1, and further such as from 1:1 to 300:1.

In a preferred embodiment, the composition according to the invention is mixed prior to be applied on hair, with an oxidizing composition.

Thus, a process according to the invention can comprise the following steps:

Applying the composition, in the presence of the oxidizing agent, on wet or dry keratin fibers;
Keeping the composition and oxidizing agent, on the fibers during approximately 1 to 60 minutes, or approximately 5 to 45 minutes;
rinsing the fibers; and
optionally washing them with shampoo, rinsing them again and then drying them.

The application of the composition according to the invention may be realized at room temperature or with the use of a warming device which is able to produce a temperature ranging from 40 to 220° C., preferably ranging from 40 to 80° C.

The cosmetic composition according to the present invention may be formulated into a multi-compartment system or kit comprising at least a first compartment and a second compartment in which the first compartment comprises the cosmetic composition according to the present invention which may optionally comprise at least one dyeing substance (8), and the second compartment comprises at least one oxidizing agent and any other optional component(s) if necessary, and the amount of fatty material(s) (component (2)) in the composition obtained by mixing the compartments may be 40% by weight or less, preferably 25% by weight or less, relative to the total weight of the composition.

The multi-compartment system may be equipped with a means for mixing and/or applying the above compositions such as a valve and a nozzle.

The composition in the form of a dispersion according to the present invention can be foamed with air, inert gas, or a mixture thereof.

According to a specifically preferred embodiment, the composition according to the present invention has the form of a temporary foam produced just before use.

According to this embodiment, the composition can be packaged in a foam dispenser. It can involve either products referred to as “aerosols” dispensed from a pressurized container by means of a propellant gas and thus forming a foam at the time of their dispensing, or compositions dispensed from a container by means of a mechanical pump connected to a dispensing head where the passage of the composition through the dispensing head transforms it into a foam in the area of the outlet orifice of such a head at the latest.

According to one embodiment, the composition can be in a “pump bottle” type foam dispenser.

These dispensers include a dispensing head for delivering the composition, a pump, and a plunger tube for transferring the composition from the container, into the head, for dispensing the product. The foam is formed by forcing the composition to pass through a material including a porous substance such as a sintered material, a filtering grid of plastic or metal, or similar structures.

Such dispensers are known to persons skilled in the art and are described in the patents: U.S. Pat. No. 3,709,437 (Wright), U.S. Pat. No. 3,937,364 (Wright), U.S. Pat. No. 4,022,351 (Wright), U.S. Pat. No. 4,1147,306 (Bennett), U.S. Pat. No. 4,184,615 (Wright), U.S. Pat. No. 4,598,862 (Rice), U.S. Pat. No. 4,615,467 (Grogan et al.), and U.S. Pat. No. 5,364,031 (Tamiguchi et al.).

According to this variant, the oxidizing agent(s) is packaged in a first container having a cap, and the alkaline agent(s) and optionally the coloring substance(s) are packaged in the second container, separate from the first, and also sealed by a closing unit. The closing unit can be a pump dispensing mechanism. The composition according to the present invention is then formed before use by mixing a composition with the oxidizing agent(s) and a composition with the coloring substance(s). For this purpose, and to limit the number of containers supplied, one of the first or second containers has an inside volume sufficient for receiving the entirety of both compositions therein. The mixture of the compositions can then be homogenized by closing this container and shaking the container. Advantageously, the container is closed directly with the dispensing head. This dispensing head comprises a mechanical pump held in a ring intended for assembly by clicking or screwing onto the neck of the container which contains the mixture. The pump comprises a pump body connected to a plunger tube in order to dispense the entirety of the mixture. The pump also comprises a pushbutton for actuating the pump body, such that with each actuation, a dose of the composition is aspirated from inside the plunger tube and ejected in foam form at the dispensing head orifice.

Preferably the containers are made of a thermoplastic material, and are made by extrusion blow molding or injection blow molding processes. In particular, the container intended for packaging the composition with the coloring substance(s) can be made of a material comprising a nonzero proportion of EVOH. For example the pump is the standard “F2-L9” model sold by the company REXAM.

According to this preferred embodiment, the purpose of the present invention is a non-aerosol device comprising the composition from the present invention.

### Examples

The present invention will be described in more detail by way of examples, which however should not be construed as limiting the scope of the present invention.

The cosmetic compositions with the formulations shown in Table 1 were prepared by mixing the components shown in Table 1. The numerical values for the amounts of the components shown in Table 1 are all based on “% by weight” as active raw materials.

TABLE 1

| | | | | Comp. |
|---|---|---|---|---|
| | Ex. 1 | Ex. 2 | Ex. 3 | Ex. 1 |
| Mineral oil | 30.00 | 30.00 | 30.00 | 30.00 |
| Ethanolamine | 5.50 | 5.50 | 5.50 | 5.50 |
| p-Phenylenediamine | — | 1.66 | — | — |
| p-Aminophenol | — | 0.43 | — | — |
| 4-Amino-2-hydroxytoluene | — | 0.09 | — | — |
| m-aminophenol | — | 0.49 | — | — |
| 2-methyl-5-hydroxyethyl- | — | 0.38 | — | — |
| aminophenol | | | | |
| Resorcinol | — | 1.88 | — | — |
| Titanium dioxide coated | 1.00 | 1.00 | 1.00 | — |
| with polydimethylsiloxane | | | | |
| Sodium metasilicate | 2.00 | 2.00 | 2.00 | — |
| Xanthane gum | 1.50 | 1.50 | 1.00 | 1.00 |
| Ascorbic acid | 1.00 | 1.00 | 1.00 | 1.00 |
| Sodium Metabasulfite | 1.00 | 1.00 | 1.00 | 1.00 |
| Pentasodium Pentetate (40%) | 2.00 | 2.00 | 2.00 | 2.00 |
| Water | Balance | Balance | Balance | Balance |

### [Evaluations]

For the cosmetic compositions according to Examples 1 to 3 and Comparative Example 1, bleaching ability, stability, and coloration ability were evaluated as follows.

### (1) Bleaching Ability

Each of the cosmetic compositions according to Example 1, Example 3, and Comparative

Example 1 was mixed with a commercial developer containing 6% of hydrogen peroxide in a weight ratio of 1:1.5 (cosmetic composition:developer), and the mixture was applied immediately onto a swatch of black Japanese hair in a weight ratio of 3:1 (cosmetic composition:hair swatch). The applied hair swatch was left for 30 minutes at 27° C. Then, It was then washed out by shampoo and dried. The color of the hair swatch was then measured by Minolta Spectrophotometer 3600-D. Calculating this value in comparison with an untreated black hair swatch and ΔE (between the color of the untreated original hair and the color of the bleached hair under the L*a*b* system) was calculated. For the evaluation of hair bleaching ability, the following criteria were used.

### Bleaching Ability Criteria

| ΔE | Bleaching Ability |
|---|---|
| Less than 5 | Low |
| Between 5-7 | Medium |
| More than 8 | High |

The results of the bleaching ability evaluation are shown below.

| | | | | Comp. |
|---|---|---|---|---|
| | Ex. 1 | Ex. 2 | Ex. 3 | Ex. 1 |
| Bleaching Ability | High | — | High | Low |

### (2) Stability

6 g of each of the cosmetic compositions according to Example 3 and Comparative Example 1 were centrifuged in a KUKOSAN H-103N Centrifugalizer for 30 seconds. After the centrifugation, the upper part of the cosmetic composition (the emulsion part if there was oil at the surface of the cosmetic composition) was subjected to microscopic observation.

Stability was checked visually, by viewing the size of the droplets via a microscope and also viewing the presence or absence at the interface of some particles. The lower the size of the droplets are and the more covered the droplets are, the more unstable the cosmetic composition is.

It was observed that the average droplet size in the cosmetic composition according to Comparative Example 1 was larger than that in the cosmetic composition according to Example 3. Moreover, the oil droplets in the cosmetic composition according to Example 3 were covered by solid particles (microscopic view), improving the stability thereof.

### (3) Coloration Ability

The cosmetic composition according to Example 2 was mixed with a commercial developer containing 6% of hydrogen peroxide in a weight ratio of 1:1.5 (cosmetic composition:developer), and the mixture was applied immediately onto a swatch of bleached Japanese (light brown) hair in a weight ratio of 3:1 (cosmetic composition:hair swatch). The applied hair swatch was left for 30 minutes at 27° C. It was then washed out by shampoo and dried. The color of the hair swatch was checked by visual observation with the eyes.

It was found that the hair swatch treated with the cosmetic composition according to Example 2 was dyed to have uniform intense dark reddish brown color.

1.-24. (canceled)

25. A cosmetic composition in the form of a dispersion, comprising:
(a) a continuous phase;
(b) at least one dispersed phase in the continuous phase;
(c) water-insoluble particles;
(d) at least one water-soluble salt; and
(e) at least one alkaline agent,
wherein the at least one dispersed phase (b) in the continuous phase is different from the component (c);
wherein components (d) and (e) are distinct compounds; and
wherein the composition optionally comprises at least one surfactant in an amount of about 1 wt % or less, relative to the total weight of the composition.

26. The cosmetic composition according to claim 25, wherein the continuous phase comprises water, and the at least one dispersed phase comprises at least one fatty material.

27. The cosmetic composition according to claim 26, wherein the at least one fatty material is in the form of a liquid at ambient temperature and under atmospheric pressure.

28. The cosmetic composition according to claim 26, wherein the at least one fatty material is chosen from oils of animal or plant origin, synthetic glycerides, fatty esters other than animal or plant oils and synthetic glycerides, fatty alcohols, liquid fatty alcohols, fatty acids, silicone oils, aliphatic hydrocarbons, and combinations thereof.

29. The cosmetic composition according to claim 26, wherein the at least one fatty material is mineral oil.

30. The cosmetic composition according to claim 26, wherein the amount of the at least one fatty material is about 50% or less by weight, relative to the total weight of the composition.

31. The cosmetic composition according to claim 25, wherein the at least one water-soluble salt is chosen from water-soluble inorganic salts, water-soluble alkaline salts, silicates, metasilicates, metasilicates of alkaline metals, carbonates, hydrogenocarbonates, phosphates, sulfates of alkaline metals, halides of ammonium, halides of alkaline metals, and combinations thereof.

32. The cosmetic composition according to claim 25, wherein the amount of the at least one water-soluble salt ranges from about 0.01% to about 50% by weight, relative to the total weight of the composition.

33. The cosmetic composition according to claim 25, wherein the water-insoluble particles comprise at least one inorganic material and/or at least one hydrophobic coating.

34. The cosmetic composition according to claim 25, wherein the water-insoluble particles are chosen from optionally surface-treated metal oxides, optionally surface-treated aluminum or transition metal oxides, optionally surface-treated silicon oxides, and combinations thereof.

35. The cosmetic composition according to claim 25, wherein the amount of the water-insoluble particles ranges from about 0.01% to about 30% by weight, relative to the total weight of the composition.

36. The cosmetic composition according to claim 25, wherein the at least one alkaline agent is chosen from inorganic compounds; ammonia; alkaline metal hydroxides; alkaline earth metal hydroxides; alkaline metal (hydrogeno)carbonates; alkaline earth metal(hydrogeno)carbonates; alkaline metal (meta)silicates; and combinations thereof.

37. The cosmetic composition according to claim 25, wherein the at least one alkaline agent is not a water-soluble salt and/or is a non-volatile agent.

38. The cosmetic composition according to claim 25, wherein the at least one alkaline agent is chosen from organic alkaline agents; non-volatile organic alkaline agents; monoamines and derivatives thereof; alkanolamines; monoethanolamine; diamines and derivatives thereof; polyamines and derivatives thereof; amino acids and derivatives thereof; oligomers of amino acids and derivatives thereof; polymers of amino acids and derivatives thereof; urea and derivatives thereof; and guanidine and derivatives thereof; and combinations thereof.

39. The cosmetic composition according to claim 25, wherein the amount of the at least one alkaline agent ranges from about 0.01% to about 15% by weight, relative to the total weight of the composition.

40. The cosmetic composition according to claim 25, further comprising at least one thickening agent and/or at least one oxidation dye and/or at least one direct dye.

41. The cosmetic composition according to claim 25, wherein the dispersion is a Pickering emulsion, optionally with an aqueous continuous phase.

42. A method of lightening or bleaching human keratin fibers, the method comprising:
applying onto the human keratin fibers in the presence of an oxidizing agent a cosmetic composition in the form of a dispersion, comprising:
(a) a continuous phase;
(b) at least one dispersed phase in the continuous phase;
(c) water-insoluble particles;
(d) at least one water-soluble salt; and
(e) at least one alkaline agent,
wherein the at least one dispersed phase (b) in the continuous phase is different from the component (c);
wherein components (d) and (e) are distinct compounds; and
wherein the composition optionally comprises at least one surfactant in an amount of about 1 wt % or less, relative to the total weight of the composition.

43. The method according to claim 42, wherein the oxidizing agent is hydrogen peroxide.

44. A method of dyeing human keratin fibers, the method comprising:
applying onto the human keratin fibers in the presence of an oxidizing agent a cosmetic composition in the form of a dispersion, comprising:
(a) a continuous phase;
(b) at least one dispersed phase in the continuous phase;
(c) water-insoluble particles;
(d) at least one water-soluble salt;
(e) at least one alkaline agent; and
(f) at least one oxidation dye and/or at least one direct dye,
wherein the at least one dispersed phase (b) in the continuous phase is different from the component (c);
wherein components (d) and (e) are distinct compounds; and
wherein the composition optionally comprises at least one surfactant in an amount of about 1 wt % or less, relative to the total weight of the composition.

45. The method according to claim 44, wherein the oxidizing agent is hydrogen peroxide.
